(19) United States
(12) Patent Application Publication
(10) Pub. No.: US 2023/0000111 A1
(43) Pub. Date: Jan. 5, 2023

(54) Method for feeding Hermetia illucens and used as for preparing composite material of pupariums

(71) Applicants: Sichuan Zhixiangyi Technology Co., Ltd, Chengdu, Sichuan (CN); Sichuan University of Science & Engineering, Zigong , Sichuan (CN); Sichuan Zhirenfa Biological Technology Co., Ltd, Zigong, Sichuan (CN)

(72) Inventors: Chi-Hui Tsou, Zigong, Sichuan (CN); Manuel Reyes De Guzman, Zigong, Sichuan (CN); Pranut Potiyaraj, Zigong, Sichuan (CN); Xuemei Zhang, Zigong, Sichuan (CN); Ya-Li Sun, Zigong, Sichuan (CN); Chen Gao, Zigong, Sichuan (CN); Xuefei Hu, Zigong, Sichuan (CN); Fei-Fan Ge, Zigong, Sichuan (CN); Juan Du, Zigong, Sichuan (CN); Chunyan Zeng, Zigong, Sichuan (CN); Tao Yang, Zigong, Sichuan (CN); Ya-Ling Lee, Zigong, Sichuan (CN); Chih-Yuan Tsou, Zigong, Sichuan (CN); Jian Chen, Zigong, Sichuan (CN); Zhijun Chen, Zigong, Sichuan (CN); Shuai Yuan, Zigong, Sichuan (CN); Yi-Hua Wen, Zigong, Sichuan (CN); Chin-San Wu, Zigong, Sichuan (CN)

(21) Appl. No.: 17/776,998

(22) PCT Filed: Nov. 13, 2020
(86) PCT No.: PCT/CN2020/128626

§ 371 Date: May 13, 2022

(30) Foreign Application Priority Data
Nov. 14, 2019 (CN) 201911116411.6

(51) Int. Cl. A23K 10/37; A23K 50/90; A01N 63/14

(52) U.S. Cl. CPC A23K 10/37; A23K 50/90; A01N 63/14

(57) ABSTRACT

This invention discloses method for feeding Hermetia illucens and used as for preparing composite material of puparium, which comprises the following steps: S1, dry Hermetia illucens pupariums and grind them into powdery, S2, adding powdery of Hermetia illucens pupariums into sodium hydroxide aqueous solution, stirring, separating and filtering, S3, adding the pupariums into hydrochloric acid solution, stirring, separating and filtering; S4, placing the pupariums into sodium hydroxide aqueous solution, stirring, separating and filtering; S5, drying the pupariums, and screening out the granular pretreated powder of the Hermetia illucens pupariums. The invention also disclose preparation method of composite material and thin film of Hermetia illucens pupariums and antibacterial and antimildew additive. The method can effectively improve the yield of chitosan in Hermetia illucens pupariums, and the prepared pupariums powder can be used for preparing polymer composite fibers and thin films of the Hermetia illucens pupariums, thus, the antibacterial effect is greatly improved. A new antibacterial and antimycotic additive can be obtained by compounding the powder of Hermetia illucens pupariums with the powder of oyster shell.

### 1. TECHNICAL FIELD

This invention belongs to the technical field of materials, and relates to the biopolymer material technology, particularly relates to method for feeding Hermetia illucens and used as for the preparation method of pupariums powder, composite materials and thin films, and novel antibacterial and antimycotic additives.

### 2. BACKGROUND

With the rapid development of materials science and the growing awareness of environmental protection, a large amount of leftover food such as kitchen waste, livestock and poultry manure and other related agricultural and livestock industries and food wastes often have problems affecting the environment, air and water quality. Therefore, some relevant scholars and experts put forward the concept of “circular economy”, in which the part of biological circulation is to make products from biodegradable raw materials, and products can be preferentially applied at different levels to maximize their value. Unavailable “biomass raw materials” can safely return to the ecological circle as nutrients after biochemical raw material extraction, biogas, composting and other procedures. Therefore, the use of agricultural resources with the concept of circular economy is a very important basis for the development of related industries of agriculture, forestry, fishery and animal husbandry. A large number of agricultural and livestock industries and food wastes can be used endlessly in the biological cycle through recycling, decomposition and reuse, thus further eliminating the concept of wastes.

A large part of China's environmental pollution comes from animal waste, which produces millions of tons of animal waste every year, such as pig manure, chicken manure, cow dung, etc., which pollutes water, soil and air, and a large amount of food waste, such as kitchen waste, vegetable residue, etc. If it can be converted into reusable resources, it can enhance the value of environmental protection and circular economy. Therefore, black hydrangea has become a major transformation of agriculture and livestock industry and food waste. The Hermetia illucens is a kind of resource insect, whose English name is Hermetia illucens, also known as Black Soldier Fly (BSF as abbreviation). The Hermetia illucens can digest a large amount of kitchen waste food and livestock manure, and transform it into new insect protein and new microbial and insect manure organic fertilizer, which can solve the problems that would otherwise affect the environment, air and water quality, and at the same time produce insect protein and fertile micro-ecological organic fertilizer, thus purifying the ecological environment and reducing the environmental burden.

The life cycle of Hermetia illucens is about 28 days, which can be roughly divided into egg stage, larval stage, pupal stage and adult stage. The egg stage is about 2 ˜ 4 days, and the egg mass is about 1 mm in diameter, which is oval, showing light yellow to milk color at the first birth, and gradually deepening at the later stage. Each egg mass contains about 500 ˜ 800 eggs. Adult spawning has suture-orientation, which is used to collect egg mass with corrugated board. The larval stage is about 15 days. The black water slug is plump, with a small yellow-black head and firm and tough epidermis. At the beginning of the first instar, it is milky white and about 1.8 mm long. There is little difference in size between the second and third instars. It is most efficient to digest food at the fourth and fifth instars, and can eat food equivalent to its own weight every day. It is called “mature larva” at Wuling stage, and the protein content is the highest at this time. Pupal stage is about 15 days, including pre-pupation stage and pupation stage. After the fifth instar, the body of the Hermetia illucens larvae gradually turns black, and tends to harden, so it stops eating and enters the pre-pupation stage. In the pre-pupation stage, the black fly does not need food, so it will look for a dry and hidden place to pupate, and it will move out of food. At the same time, it has the characteristics of rotation-avoiding optical activity and seam-approaching. After pupation, the pupa shell is dark brown, which is the peripupa formed by the molting of the last larvae, and the pupa body can be seen when cut open. Then, the Hermetia illucens will emerge as an adult, and the adult black water slug has only about 5 days to live. After the pupa emerges as an adult, it only feeds on the water and sap on the leaves, and after the flight mating, the female worm lays eggs.

The Hermetia illucens can circulate for 7-10 generations a year. In a suitable environment, a generation can be completed in 28 ˜ 35 days. A pair of Hermetia illucens can lay nearly 1,000 eggs. From eggs to mature larvae, the individual growth of black hydra is nearly 4,000 times. The worm itself in each stage can be used as an important economic value. The dried and crushed black hydra larvae can be made into health products for aquatic products and livestock and poultry breeding, according to the research, the content of amino acid, crude fat and calcium in the larval powder of black hydra is very high, which is a suitable additive for pig feed and fish feed. The dung “carbon fertilizer of black fly” is a kind of high-quality organic fertilizer and soil conditioner, with uniform particles and no peculiar smell, and its water retention and air permeability are 2-3 times higher than that of common soil. It contains 18 kinds of amino acids, contains 42.2% of organic matter, and has 200,000-200 million beneficial bacteria per gram. Only a small amount of carbon fertilizer of Hermetia illucens can achieve the highest benefit and effectively increase the soil.

Although the Hermetia illucens has many additional economic values mentioned above, such as dried and crushed products of Hermetia illucens larvae used in pig feed, and black fly manure carbon fertilizer is a kind of high-quality organic fertilizer and soil modifier, a large number of pupal shells produced during the growth of Hermetia illucens have not been further utilized. According to the research, the Hermetia illucens contains a large amount of protein, lipid and soil conditioner. Most of the oil performance parameters of bio-diesel produced from the oily animal fat contained in the black fly can meet the EU bio-diesel standard, which shows that the black fly can become a new material source of bio-diesel. However, there are abundant chitin and antibacterial peptides in the pupal shell of the Hermetia illucens, which has not been widely used at present. Chitin, also known as chitin, is a special glycoprotein with similar chemical structure to natural cellulose, and can be effectively and widely used in medicine, bioengineering, light industry, food and other fields. The reason why the black water slug can survive in a large amount of kitchen waste and fertilizer without getting sick is that the black water slug itself has a unique immune system, which can synthesize a large number of antibacterial peptides in the body to kill invading bacteria. Antibacterial peptides are substances that can resist and inhibit Escherichia coli, Staphylococcus aureus and Salmonella, and insect antibacterial peptides are natural products, which are not easy to produce drug resistance. These antibacterial peptides have antibacterial ability against bacteria and fungi, viruses and tumors.

To sum up, if a large amount of biological wastes such as the shell of Hermetia illucens can be recovered and treated as polymer matrix contents, so as to reduce the cost of the substrate, and at the same time strengthen the performance and efficacy of the polymer matrix, such as non-toxicity, antibacterial, good mechanical properties and elongation, the economic value of recycling waste shell pupa of black hydra can be greatly improved, and a high-efficiency and low-cost polymer composite material of black hydra pupa can be formed.

### 3. SUMMARY

In view of this, the inventor studied and improved the above-mentioned needs and deficiencies, and developed a method which can effectively utilize the polymer of the Hermetia illucens pupariums and recycle it for processing and synthesis, so as to provide a non-toxic, antibacterial, high-molecular composite material of the Hermetia illucens pupariums with good mechanical properties and elongation rate, and provide consumers with more innovative and unique products with excellent quality, which is the motive of the invention.

Feeding method of Hermetia illucens is to use starchy kitchen waste food or mixed starchy kitchen waste food, distiller's grains and corn straw (inner).

Preparation method of Hermetia illucens pupariums powder comprises the following steps:

S1, dry Hermetia illucens pupariums and grind them into powdery,

S2, adding powdery of Hermetia illucens pupariums into sodium hydroxide aqueous solution, stirring, separating and filtering,

S3, adding the pupariums into hydrochloric acid solution, stirring, separating and filtering,

S4, placing the pupariums into sodium hydroxide aqueous solution, stirring, separating and filtering,

S5, drying the pupariums, and screening out the granular pretreated powder of the Hermetia illucens pupariums.

Preparation method of composite material of Hermetia illucens pupariums is to mix the pretreated powder of the Hermetia illucens pupariums with a polymer substrate according to the weight ratio of 0.1-25%, and then heating and melting the mixture.

Preferably, after melting the mixture and then wire drawing to form fibrous composite material.

Preferably, the polymer base material is any one or a mixture of a plurality of polyterephthalate, polyamide, polyolefin, polyurethane, polymethylmethacrylate, polyvinylpyrrolidone, cellulose, polyvinyl alcohol and biodegradable polymer materials.

The preparation method of of compound thin film of Hermetia illucens pupariums is to add the pre-treated powder of the Hermetia illucens pupariums into a liquid substrate, stirring at high speed to prepare thin film, and drying the film to obtain the Hermetia illucens pupariums polymer composite film.

Preferably, the method for preparing the film is tape casting.

Preferably, the liquid substrate is any one of polyvinyl alcohol aqueous solution, aqueous polyurethane or carboxymethyl cellulose aqueous solution.

A novel kind of antibacterial and antimildew additive of this invention is obtained by mixing the Hermetia illucens pupariums pretreatment powder with oyster shell powder, and the weight content of oyster shell powder is not less than 20%.

By adopting that method for feeding the Hermetia illucens, the yield of chitosan in the Hermetia illucens pupariums can be effectively improved.

The pupariums powder prepared by the method can be used for preparing the fiber and thin film of the Hermetia illucens pupariums polymer composite material, and the antibacterial effect of the obtained polymer composite material is greatly improved.

Due to the addition of Hermetia illucens pupariums powder particles in polymer matrix, the biodegradation efficiency can be improved, the cell activity of polymer can be improved, and the biotoxicity can be reduced. When the polymer composite material of Hermetia illucens pupariums is abandoned and buried, it can promote the conversion rate of microbial decomposition after being buried in soil, and can be used as the growth nutrient of crops. Meanwhile, the invention adopts the black hydra pupa shell which is originally discarded as waste as the raw material, thereby reducing a large amount of cost.

### 4. BRIEF DESCRIPTION OF THE FIGURES

FIG. 1 is a schematic diagram of a specific embodiment of the black hydra pupa shell composite material after forming fibers.

FIG. 2 is a schematic diagram of a specific manufacturing process of the black water fly pupa shell powder according to the present invention.

FIG. 3 is a schematic diagram of the fiber making process of the composite material of black hydra pupa shell according to the present invention.

Wherein:

1—Hermetia illucens pupariums polymer composite fiber

2—Polymer substrate

20—polymer composite material

3—Hermetia illucens pupariums powder.

30—Hermetia illucens pupariums

301—The pupariums powder of the black Hermetia illucens treated in S2.

302—The pupariums powder of the Hermetia illucens treated in S3.

303—The pupariums powder of the Hermetia illucens treated in S4.

4—Grinding machine.

5—Mixer.

51—Low concentration sodium hydroxide solution.

52—Hydrochloric acid solution.

53—High concentration sodium hydroxide solution.

6—Vibration screening machine.

7—Macromolecular composite material of Hermetia illucens pupariums.

### 5. DESCRIPTION

With reference to the accompanying drawings, the specific embodiments of the present invention will be further described in detail below.

### Embodiment 1

Five groups of Hermetia illucens were fed separately with material described as below:

(1) Starch waste residue, such as starch kitchen waste such as noodles, bread, steamed bread and leftovers;

(2) Distiller's grains

(3) Straw

(4) Meat

(5) Livestock manure

The Hermetia illucens pupariums left by the adults of the black hydra in five feeding ways were collected.

The Hermetia illucens pupariums powder was prepared in the following manner:

S1, drying Hermetia illucens pupariums, and grinding into pow by grinding machine,

S2, adding Hermetia illucens pupariums powder into 3.5 wt % low-concentration sodium hydroxide solution 51, stirring by a mixer 5, and then separating and filtering. The main function of this step is to remove the protein on the surface of pupariums.

S3, adding the Hermetia illucens pupariums powder obtained in step S2 into 12 equivalent concentration of hydrochloric acid solution 52, stirring, and separating and filtering.

The main function of this step is to remove the mineral salt components in the pupariums.

S4, putting the Hermetia illucens pupariums obtained in step S3 into 30 wt % high-concentration sodium hydroxide solution 53, stirring, separating and filtering.

S5, drying the Hermetia illucens pupariums obtained in the step S4, crushing and grinding the dried black hydra pupa shell into particles with required particle size or Hermetia illucens pupariums pretreatment powder.

By adopting the above method, the preparation of pupariums powder can be quickly and simply completed, and high-purity chitosan can be extracted, and a relatively high yield can be obtained compared with the traditional method.

Table 1 shows the yield of chitosan in the Hermetia illucens pupariums powder prepared by the above method and the traditional method:

TABLE 1

| | The method of | Traditional |
|---|---|---|
| Food types of Hermetia illucens | this invention(%) | method(%) |
| Starchy kitchen waste such as | 38.8 | 30.4 |
| noodles, bread, steamed bread and | | |
| leftovers (a) | | |
| Distiller's grains (b) | 28.3 | 21.5 |
| Corn Straw (c) (skin and stalk | 23.9 | 17.3 |
| attached | | |
| Meat kitchen waste such as pork. | 16.6 | 11.7 |
| Beef, chicken (f) | | |
| Livestock manure (g) | 8.9 | 4.2 |

From Table 1, it can be seen that different food sources will affect the yield of chitosan extracted from the pupa shell of the Hermetia illucens, among which the food source of starch waste residue is the best, corn straw (inner) (d) Inner stalk of straw is the second, distiller's grains is the third, and the yield of livestock manure is the lowest. Compared with the traditional preparation method, the yield of chitosan obtained by treating the pupa shells of the hydra in the present invention shows that the yield of the new process of the present invention is increased by about 5-8% compared with that of the traditional process under the same food source. Compared with the traditional process, the new process greatly reduces the process flow, the process is not complicated, and it is more suitable for industrialization.

### Embodiment 2

Melt-blend the Hermetia illucens pupariums powder prepared in embodiment 1 (a) with different polymer substrates, such as polypropylene, polyamine, polylactic acid, polyethylene terephthalate, etc., to prepare different Hermetia illucens pupariums polymer composite materials, so that the Hermetia illucens pupariums 3 can be uniformly distributed in the polymer composite material 20. The specific method is to mix the polymer substrate powder with the black fly pupa shell powder uniformly.

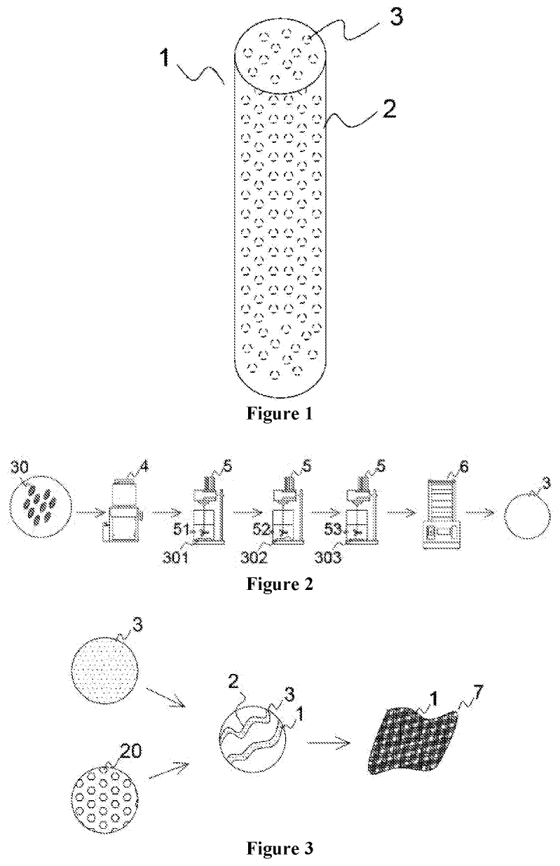

Then, through spinning by thermoplastic equipment, etc., the polymer composite fiber 1 of Hermetia illucens pupariums as shown in FIG. 1 can be produced, and the obtained composite fiber can be further made into a non-toxic, safe, antibacterial, breathable macromolecular composite material of Hermetia illucens puparium 7 with good mechanical properties and elongation, as shown in FIG. 3.

When the polymer substrate does not contain the Hermetia illucens pupariums powder, it has no antibacterial effect. When the mass ratio of Hermetia illucens pupariums powder in the mixture reaches 0.1%, the composite material begins to show antibacterial effect. When the mass ratio of Hermetia illucens pupariums powder in the mixture reaches 20%, the antibacterial rate of all the composite materials except polypropylene-based composite material is 99.6%. The relative cell proliferation rate, which reflects the cell activity, is also greatly improved with the increase of the content of the Hermetia illucens pupariums powder. The specific test parameters are shown in Table 2.

TABLE 2

| | Content of | | |
|---|---|---|---|
| | Hermetia | | |
| | illucens | | Relative cell |
| Types of polymer | pupariums | Antibacterial | proliferation |
| substrates | powder(%) | ratio(%) | rate(%) |
| Polypropylene | 0 | 0 | 103.1 |
| Polypropylene | 0.1 | 7.3 | 107.1 |
| Polypropylene | 0.5 | 88.8 | 111.3 |
| Polypropylene | 20 | 99.6 | 178.9 |
| Polypropylene | 0 | 0 | 116.2 |
| Polypropylene | 0.1 | 14.4 | 118.1 |
| Polypropylene | 0.5 | 95.1 | 122.3 |
| Polypropylene | 20 | 100 | 209.1 |
| Polylactic acid | 0 | 0 | 258.9 |
| Polylactic acid | 0.1 | 19.2 | 263.6 |
| Polylactic acid | 0.5 | 91.7 | 289.5 |
| Polylactic acid | 20 | 100 | 333.3 |
| Polyethylene glycol | 0 | 0 | 210.2 |
| terephthalate | | | |
| Polyethylene glycol | 0.1 | 11.8 | 211.4 |
| terephthalate | | | |
| Polyethylene glycol | 0.5 | 88.3 | 224.9 |
| terephthalate | | | |
| Polyethylene glycol | 20 | 100 | 299.3 |
| terephthalate | | | |

### Embodiment 3

Dissolving 14.925 g and 12 g of carboxymethyl cellulose in 100 ml aqueous solution respectively, and 0.075 g and 3 g of Hermetia illucens pupariums powder prepared in embodiment 1 (a) were added to the aqueous solution of carboxymethyl cellulose respectively. The powder was dispersed in carboxymethyl cellulose by stirring at a high speed of 1000 rpm, and several films were scraped with a scraper with a thickness of 600 um, and dried in an oven for 8 hours to prepare composite film of Hermetia illucens pupariums with content of Hermetia illucens pupariums reaches 0.5% and 20% respectively. The method for preparing the film can also be tape casting, in which the powder is melted and plasticized from an extruder, extruded through a slit die, so that the melt is tightly attached to a cooling roller, and the sheet is made by stretching, trimming, coiling and other processes.

The specific test performance is shown in Table 3. The carboxymethyl cellulose film without adding the powder of the black fly pupa shell has no antibacterial effect, and the antibacterial rate increases with the increase of the powder content of the Hermetia illucens pupariums. As the Hermetia illucens pupariums is a natural biological material, its cell activity also increases with the increase of the content of the Hermetia illucens pupariums.

TABLE 3

| | Content of | | |
|---|---|---|---|
| | Hermetia | | |
| | illucens | | Relative cell |
| | pupariums | Antibacterial | proliferation |
| | powder(%) | ratio(%) | rate(%) |
| Carboxymethylcellulose | 0 | 0 | 233.1 |
| Carboxymethylcellulose | 0.1 | 13.3 | 237.1 |
| Carboxymethylcellulose | 0.5 | 93.9 | 265.5 |
| Carboxymethylcellulose | 20 | 100 | 302.4 |

### Embodiment 4

Polyol polybutylene glycol adipic acid, polyether glycol and internal emulsifier dimethylolpropionic acid are added into a four-mouth separated reaction tank equipped with a fixed-speed stirrer, a temperature controller and the like, and a mixture is obtained after reacting at 105° C. for 20 minutes, and water is removed by separation; Then, when the temperature is lowered to 80° C., the pupa shell of black hydra is added, and hydrogenated phenyl methane diisocyanate, 2,6-pyridine dimethanol and several drops of dibutyltin dilaurate catalyst diluted to 5% with DMAC (dimethylacetamide) are added at the rotating speed of 300 rpm.

Reacting in a reactor for 2.5 hours to obtain a prepolymer with NCO (isocyanate) groups at the terminal, then dispersing into aqueous PU dispersion by acetone processing method; When the temperature is reduced to 55° C., triethylamine is added for neutralization reaction for 20 minutes, then, increasing the rotating speed to 1000 rpm, adding deionized water to a concentration of 65 wt %, and stirring until the polymer is uniformly dispersed in water. Finally, the chain extender ethylenediamine is added for chain extension reaction for 30 minutes. The obtained product is pyridine-containing waterborne polyurethane with solid content of 35 wt %.

According to the weight of 0.1% and 20% of the solid content of the waterborne polyurethane, the powder of the Hermetia illucens pupariums prepared in embodiment 1 (a) was added into the waterborne polyurethane containing pyridine for high-speed stirring at 1000 rpm, and then several films were scraped by a scraper with a thickness of 600 um, and dried in an oven for 8 hours to prepare the black fly pupa shell composite film. The antibacterial rate and the relative cell proliferation rate of the product are tested as shown in Table 4.

TABLE 4

| | Content of | | |
|---|---|---|---|
| | Hermetia | | |
| | illucens | | Relative cell |
| | pupariums | Antibacterial | proliferation |
| | powder(%) | ratio(%) | rate(%) |
| Polyurethane | 0 | 0 | 174.5 |
| Polyurethane | 0.1 | 9.1 | 174.9 |
| Polyurethane | 0.5 | 90.2 | 192.3 |
| Polyurethane | 20 | 100 | 235.7 |

### Embodiment 5

Dissolving 29.85 g and 24 g of polyvinyl alcohol in 200 ml aqueous solution at 85° C., 0.15 g and 6 g of Hermetia illucens pupariums powder prepared in embodiment 1 (a) were added to the aqueous solution of polyvinyl alcohol, and the powder was dispersed in carboxymethyl cellulose by stirring at 500 rpm. Several films were scraped with a scraper with a thickness of 600 um, and dried in an oven at 80° C. for 10 hours, the composite films of Hermetia illucens pupariums with the Hermetia illucens pupariums content of 0.1% and 20% respectively were prepared.

The specific test performance is shown in Table 3. The polyvinyl alcohol film without adding Hermetia illucens pupariums powder prepared in embodiment 1 (a) has no antibacterial effect, and the antibacterial rate increases with the increase of the powder content of the Hermetia illucens pupariums. As Hermetia illucens pupariums is a natural biological material, its cell activity also increases with the increase of the content of the Hermetia illucens pupariums powder.

TABLE 5

| | Content of | | |
|---|---|---|---|
| | Hermetia | | |
| | illucens | | Relative cell |
| | pupariums | Antibacterial | proliferation |
| | powder(%) | ratio(%) | rate(%) |
| Polyvinyl alcohol | 0 | 0 | 238.7 |
| Polyvinyl alcohol | 0.1 | 7.3 | 241.2 |
| Polyvinyl alcohol | 0.5 | 94.3 | 256.8 |
| Polyvinyl alcohol | 20 | 100 | 311.5 |

The foregoing are all the preferred embodiments of the present invention. If the preferred embodiments in each preferred embodiment are not obviously contradictory or based on a certain preferred embodiment, each preferred embodiment can be used in any superposition and combination. The embodiments and the specific parameters in the embodiments are only for clearly describing the inventor's invention verification process, and are not used to limit the patent protection scope of the present invention. The patent protection scope of the present invention shall still be subject to its claims. Any equivalent structural changes made by using the contents of the specification and drawings of the present invention shall be equally applicable.

### Embodiment 6

The Hermetia illucens pupariums gel was prepared by dissolving the Hermetia illucens pupariums powder prepared in embodiment 1 (a) in 1.8% acetic acid aqueous solution. The calcined oyster shell powder was added into the Hermetia illucens pupariums with the ratio of 20%, 40%, 60% and 80%, respectively. First, it was vibrated and dispersed for 10 minutes with high-sound cell breaker to avoid agglomeration of oyster shell powder. According to the antibacterial and anti-mildew tests of oyster shell powder with different proportions, Table 6 shows that although the Hermetia illucens pupariums has good antibacterial performance, it has no good anti-mildew effect. With the increase of oyster shell powder content, its anti-mildew effect will be better. This novel composite bio-based powder can be used as an excellent antibacterial and antimycotic additive.

TABLE 6

| | | | Relative cell |
|---|---|---|---|
| | Content of oyster | Antibacterial | proliferation |
| | shell powder (%) | ratio(%) | rate(%) |
| Hermetia illucens | 0 | 99.9 | 16.8 |
| pupariums | | | |
| Hermetia illucens | 20 | 99.9 | 63.5 |
| pupariums | | | |
| Hermetia illucens | 40 | 99.9 | 97.3 |
| pupariums | | | |
| Hermetia illucens | 60 | 99.9 | 99.9 |
| pupariums | | | |
| Hermetia illucens | 80 | 99.9 | 99.9 |
| pupariums | | | |

The above are various embodiments of the present invention, and their descriptions are more specific and detailed, but they cannot be understood as limiting the patent scope of the present invention. It should be pointed out that for those of ordinary skill in the field, without departing from the concept of the present invention, several modifications and improvements can be made, which all belong to the protection scope of the present invention. Therefore, the scope of protection of the present invention shall be subject to the appended claims.

1. Feeding method of Hermetia illucens, is characterized in that use starchy kitchen waste food or corn straw (inner) or mixed starchy kitchen waste food, distiller's grains and corn straw (inner) as feeds, and used as for preparation method of Hermetia illucens pupariums powder, is characterized in comprising the following steps:
S1, dry Hermetia illucens pupariums and grind them into powdery,
S2, adding powdery of Hermetia illucens pupariums into sodium hydroxide aqueous solution, stirring, separating and filtering,
S3, adding the pupariums into hydrochloric acid solution, stirring, separating and filtering,
S4, placing the pupariums into sodium hydroxide aqueous solution, stirring, separating and filtering,
S5, drying the pupariums, and screening out the granular pretreated powder of the Hermetia illucens pupariums.

2. (canceled)

3. Preparation method of composite material of Hermetia illucens pupariums is characterized in that the method comprises the following steps of: mixing the pretreated powder of the Hermetia illucens pupariums with a polymer substrate according to the weight ratio of 0.1-25%, and then heating and melting the mixture.

4-8. (canceled)

9. A novel kind of antibacterial and antimildew additive is characterized in that the Hermetia illucens pupariums pretreatment powder is mixed with oyster shell powder, and the weight content of oyster shell powder is not less than 20%.
